(19) United States
(12) Patent Application Publication
(10) Pub. No.: US 2024/0100125 A1
(43) Pub. Date: Mar. 28, 2024

(54) SYNERGISTIC EFFECT OF A FXR AGONIST AND IFN FOR THE TREATMENT OF HBV INFECTION

(71) Applicants: ENYO PHARMA, LYON (FR); INSERM (INSTITUT NATIONAL DE LA SANTÉ ET DE LA RECHERCHE MÉDICALE), PARIS (FR); CENTRE NATIONAL DE LA RECHERCHE SCIENTIFIQUE, PARIS (FR); CENTRE LEON BÉRARD, LYON (FR); UNIVERSITÉ CLAUDE BERNARD LYON 1, VILLEURBANNE (FR)

(72) Inventors: RAPHAËL DARTEIL, LYON (FR); PIETRO SCALFARO, FIESCH (CH); JACKY VONDERSCHER, SENOUILLAC (FR); ELISE ROY, LES FINS (FR); DAVID DURANTEL, GENAS (FR)

(21) Appl. No.: 18/272,116

(22) PCT Filed: Jan. 13, 2022
(86) PCT No.: PCT/EP2022/050589

§ 371 Date: Jul. 13, 2023

(30) Foreign Application Priority Data
Jan. 14, 2021 (EP) 21305035.4

(51) Int. Cl. A61K 38/21; A61K 31/4162; A61K 31/42; A61K 31/46; A61K 31/575; A61K 45/06; A61K 47/60; A61P 31/20

(52) U.S. Cl. CPC A61K 38/212; A61K 31/4162; A61K 31/42; A61K 31/46; A61K 31/575; A61K 45/06; A61K 47/60; A61P 31/20

(57) ABSTRACT

The present invention relates to a synergistic combination of an FXR agonist and interferon for the treatment of hepatitis B.

### FIELD OF THE INVENTION

The present invention relates to a method for treating Hepatitis B infection.

### BACKGROUND OF THE INVENTION

Hepatitis B remains a major worldwide public health problem with over 350 million of chronically-infected people despite extensive vaccination programs. Chronic hepatitis B evolves towards life threatening complications including liver cirrhosis and cancer. Current therapeutic regimens are long term treatments (e.g., polymerase inhibitors, life long; pegylated interferons up to one year) and fail to cure HBV as they do not target the virus reservoir. HBV functional cure remains a major unmet medical need.

The primary goal of treatment for chronic hepatitis B (CHB) is to permanently suppress HBV replication and prevent or improve liver disease. Seven drugs are currently available for treatment of CHB infection-conventional interferon (IFN), pegylated interferon and direct antiviral agents. The direct antivirals (nucleos/tide analogues) belong to three classes: L-nucleosides (lamivudine, telbivudine and emtricitabine); deoxyguanosine analogs (entecavir) and nucleoside phosphonates (adefovir and tenofovir) which directly interfere with HBV DNA replication, primarily as chain terminators. The key limitations for interferon treatment are major side-effects, low rate of HBV DNA suppression and low rate of ALT normalization; key limitations of the treatment with direct antivirals are: development of resistance; rebound of HBV replication after stopping therapy requiring prolonged, life-long therapy, very low rate of HBsAg clearance, increasing the risk of adverse events with prolonged, life-long therapy. Importantly, current direct antivirals repress the reverse transcription of the pre-genomic viral RNA into the genomic DNA. They thus act downstream to the formation of the covalently closed circular DNA (cccDNA) that is formed after virus entry into hepatocytes. cccDNA reside in the cell nucleus as additional minichromosomes that are transcribed into viral mRNAs and transmitted to daughter cells when hepatocytes divide. Current direct antivirals have no or very little effect on the HBV cccDNA reservoir and the expression of the viral genes. Thus, the currently available treatments are suboptimal and may be associated with severe side effects.

WO 2015/036442 discloses the interest of FXR agonist for decreasing HBV replication. EYP001 is a synthetic non-steroidal, non-bile acid FXR agonist with a good tolerability profile. EYP001 is an orally bioavailable small molecule currently evaluated in phase Ib in patients with chronic hepatitis B. Contrary to lifelong standards of care that target essentially virus replication, EYP001 is targeting the cccDNA (‘virus reservoir’), therefore aiming for HBV real cure. Erken et al (2018, Journal of Hepatology, 68, Suppl 1, S488-S489) discloses that EYP001 reduces HBV viral load in chronic hepatitis B patients. Joly et al (2017, Journal of Hepatology, 66, Suppl 1, SAT-158) discloses that EYP001 and nucleosides analogue can be safely used in healthy individuals and have additive effects on HBV reduction/elimination in cell culture.

Several combined therapies with IFN and nucleo(s/t)ide analogs have been studied: namely IFN-α and a drug selected from lamivudine, adefovir, telbuvirine, entecavir and tenofovir (Woo et al, 2017, Ann Transl Med, 5, 159). Almost all combinations failed to show any benefit. Indeed, only one combination with tenofovir achieves higher rates of HBsAg (hepatitis B surface Antigen) loss with a percentage of less than 10%. However, such a low rate of HBsAg loss makes cure remaining elusive.

However, there is always a need for better therapies to meet the treatment goals in HBV infection, in particular CHB infection.

### SUMMARY OF THE INVENTION

The inventors surprisingly identified that FXR agonists have a synergistic effect with interferon for the treatment of hepatitis B, especially on pre-genomic viral RNA, a marker of viral replication, and on HBcrAg, a core related antigen which is a serum marker of chronic hepatitis B. More particularly, EYP001 (Vonafexor) and IFN have a synergistic effect on the reduction of cccDNA transcription. These effects were observed after only 4 weeks of treatment which is a very short period of time, whereas neither EYP001 and nor IFN when used alone do not show a significant effect at the same dose and after the same period of time. In addition, surprisingly, the synergistic effect is at least two-fold stronger when EYP001 is administered once a day in comparison to an administration twice a day with the same daily dosing. Similarly, a synergistic effect has been observed with another FXR agonist, namely GW4064, on intracellular HBV RNA level, as well as HBeAg and HBsAg secretion and with other FXR agonists, namely Tropifexor, Nidufexor, and Ocaliva (OCA), on HBsAg secretion in two different models of HBV infected hepatocytes.

Therefore, the present invention relates to the synergistic combination of an FXR agonist and IFN for use in the treatment of hepatitis B infection, especially chronic hepatitis B.

The present invention relates to an FXR agonist or a pharmaceutic composition comprising it for use in combination with interferon alpha (IFN-α) or a pegylated form thereof for the treatment of hepatitis B virus infection, especially chronic hepatitis B, wherein the FXR agonist and IFN-α are used so as to obtain a synergistic effect for decreasing the HBV replication. Optionally, the FXR agonist is not EYP001.

It also relates to the use of an FXR agonist or a pharmaceutical composition comprising it for the manufacture of a drug for the treatment of hepatitis B virus infection, especially chronic hepatitis B, in combination with interferon alpha (IFN-α) or a pegylated form thereof, wherein the FXR agonist and IFN-α are used so as to obtain a synergistic effect for decreasing the HBV replication. It further relates to a method for treating a hepatitis B virus infection, especially chronic hepatitis B, in a subject in need thereof, comprising administering a therapeutically effective or sub-therapeutic amount of an FXR agonist and administering a therapeutically effective or sub-therapeutic amount of interferon alpha (IFN-α) or a pegylated form thereof, wherein the FXR agonist and IFN-α are administered so as to obtain a synergistic effect for decreasing the HBV replication. Optionally, the FXR agonist is not EYP001.

Optionally, the FXR agonist is selected from the group consisting of LIN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502. In a particular aspect, the FXR agonist is selected from the group consisting of LIN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502, or any pharmaceutically acceptable salt thereof. In a more specific aspect, the FXR agonist is selected from the group consisting of Tropifexor, Nidufexor, Ocaliva and GW4064 or any pharmaceutically acceptable salt thereof.

Optionally, the FXR agonist is to be administered at a sub-therapeutic amount.

In one aspect, the FXR agonist is to be administered once a day. In another aspect, the FXR agonist is to be administered twice a day.

In one aspect, the IFN-α is IFN-α2a, IFN-α2b or a pegylated form thereof. Preferably, IFN-α or a pegylated form thereof is to be administered by subcutaneous route once a week. Optionally, IFN-α or a pegylated form thereof can be administered at a sub-therapeutic amount.

In one particular aspect, both the FXR agonist and IFN-α or a pegylated form thereof are to be administered at sub-therapeutic amounts.

In one aspect, the FXR agonist and IFN-α or a pegylated form thereof are to be administered during a period of time from 5 6, 7 or 8 weeks to 52 weeks.

In one aspect, the FXR agonist and IFN-α or a pegylated form thereof are to be used in combination with at least one additional active ingredient. More specifically, the at least one additional active ingredient is a polymerase inhibitor selected from the group consisting of L-nucleosides, deoxyguanosine analogs and nucleoside phosphonates. In a very specific aspect, the at least one additional active ingredient is selected from the group consisting of lamivudine, telbivudine, emtricitabine, entecavir, adefovir and tenofovir.

### BRIEF DESCRIPTION OF THE FIGURES

FIG. 1: HBV pgRNA (log 10 copies/mL) changes from baseline after a 4 week anti-HBV treatment course of FXR agonist EYPOO1a or placebo in combination with interferon in chronically infected previously untreated HBV patients. PBO: placebo. peg-IFN: pegylated interferon alpha2a. 150 BID: 150 mg twice daily. 300 QD: 300 mg once daily. pgRNA: pregenomic ribonucleic acid. Black columns are changes at end of treatment Day 29. Grey columns are changes one week after end of treatment at Day 35. *p=0.04

FIG. 2: HBcrAg (log 10 μl/mL) changes from baseline after a 4 week anti-HBV treatment course of FXR agonist EYPOO1a or placebo in combination with interferon in chronically infected previously untreated HBV patients. PBO: placebo. peg-IFN: pegylated interferon alpha2a. HBcrAg: hepatitis B core-related antigen. Black columns are changes at end of treatment Day 29. Grey columns are changes one week after end of treatment at Day 35.

FIG. 3. Synergistic effect of FXR agonist and IFN-alpha on HBV replication in infected primary human hepatocytes (PHH). Freshly prepared and seeded PHH were infected with HBV at a multiplicity of infection of 250 GE/cell. From day 4 to 10 post-infection, cells were treated with at 1 or 10 uM, +/−IFN-alpha at 100 IU/mL, or vehicle (NT), or IFN-alpha at 100 IU/mL alone. Cells and supernatants were harvested at day 10 for intracellular HBV RNA, viremia, and secreted antigens (HBsAg and HBeAg) quantification. Results are the mean+/−SD of one experiment performed with three biological replicates.

FIG. 4. Synergistic effect of FXR agonists and IFN-alpha on HBsAg secretion in HBV infected primary human hepatocytes (PHH). Freshly prepared and seeded PHH were infected with HBV at a multiplicity of infection of 250 GE/cell. From day 4 to 10 post-infection, cells were treated with Vonafexor at 10 uM, or Nidufexor at 10 uM, or Tropifexor at 1 uM, or OCA at 10 uM, or GW4064 at 10 uM, +/−IFN-alpha (IFN) at 100 IU/mL, or vehicle, or IFN-alpha at 100 IU/mL alone. Supernatants were harvested at day 10 for secreted HBs antigen (HBsAg) quantification. Results are the mean+/−SEM of five experiments performed with three replicates.

FIG. 5. Synergistic effect of FXR agonists and IFN-alpha on HBsAg secretion in HBV infected dHepaRG cells. Differentiated HepaRG (dHepaRG) cells were infected with HBV at a MOI of 250 GE/cell. From day 7 to day 14 post-HBV infection, cells were treated with Vonafexor at 10 uM, or Nidufexor at 10 uM, or Tropifexor at 1 uM, or OCA at 10 uM, or GW4064 at 10 uM, +/−IFN-alpha at 25 IU/mL, or vehicle, or IFN-alpha at 25 IU/mL alone. Supernatants were harvested at day 14 for secreted HBs antigen (HBsAg) quantification. Results are the mean+/−SEM of three experiments performed with three replicates.

### DETAILED DESCRIPTION OF THE INVENTION

The inventors observed that a combined treatment of an FXR agonist with IFN-α surprisingly leads to a synergistic effect on chronic hepatitis B. Therefore, a therapeutic benefit can be obtained for the patient by using the synergistic combination of an FXR agonist with IFN-α.

### Definition

The term “FXR” refers to the farnesoid X receptor, which is a nuclear receptor that is activated by supraphysiological levels of farnesol (Forman et al., Cell, 1995, 81, 687-693). FXR, is also known as NR1H4, retinoid X receptor-interacting protein 14 (RIP14) and bile acid receptor (BAR). Containing a conserved DNA-binding domain (DBD) and a C-terminal ligand-binding domain (LBD), FXR binds to and becomes activated by a variety of naturally occurring bile acids (BAs), including the primary bile acid chenodeoxycholic acid (CDCA) and its taurine and glycine conjugates. Upon activation, the FXR-RXR heterodimer binds the promoter region of target genes and regulates the expression of several genes involved in bile acid homeostasis. Hepatic FXR target genes fall into two main groups. The first group functions to decrease hepatic bile acids concentrations by increasing export and decreasing their synthesis. The second group of FXR target genes such as the phospholipid transport protein PLTP and apolipoproteins modulates lipoprotein levels in the serum and decreases plasma triglyceride concentration. For a more detailed list of FXR-regulated genes, see, e.g., WO 03/016288, pages 22-23. U.S. Pat. No. 6,005,086 discloses the nucleic acid sequence coding for a mammalian FXR protein. The human polypeptide sequences for FXR are deposited in nucleotide and protein databases under accession numbers NM_005123, Q96RI1, NP_005114 AAM53551, AAM53550, AAK60271.

In this specification, the term “FXR agonist” has its general meaning in the art and refers in particular to compounds that function by targeting and binding the farnesoid X receptor (FXR) and which activate FXR by at least 40% above background in the assay described in Maloney et al. (J. Med. Chem. 2000, 43:2971-2974).

In some embodiments, the FXR agonist of the invention is a selective FXR agonist. As used herein, the term “selective FXR agonist” refers to an FXR agonist that exhibits no significant cross-reactivity to one or more, ideally substantially all, of a panel of nuclear receptors consisting of LXRα, LXRβ, PPARα, PPARγ, PPARδ, RXRα, RARγ, VDR, PXR, ERα, ERβ, GR, AR, MR and PR. Methods of determining significant cross-reactivity are described in J. Med. Chem. 2009, 52, 904-907.

As used herein, the terms “treatment”, “treat” or “treating” refer to any act intended to ameliorate the health status of patients such as therapy, prevention, prophylaxis and retardation of a disease. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease. More particularly, the term “treating”, or “treatment”, means alleviating HBV infection, arresting disease development, and/or removing HBV by administering the composition.

More particularly, the treatment of hepatitis B infection, especially chronic hepatitis B, is shown by a decrease of HBV replication. The HBV replication can be assessed by determining at least one of HBeAg levels, HBsAg levels, HBcrAg levels, pre-genomic RNA (HBV pgRNA) levels, pre-core RNA levels, relaxed circular DNA (HBV rcDNA) levels, HBV cccDNA levels or HBV DNA levels in the subject. HBsAg loss and seroconversion are generally the goal for clinical cure. By decreasing, it is meant that the level in at least one of HBeAg levels, HBsAg levels, HBcrAg levels, pre-genomic RNA (HBV pgRNA) levels, pre-core RNA levels, relaxed circular DNA (HBV rcDNA) levels, HBV cccDNA levels and HBV DNA levels is decreased in comparison with the absence of treatment.

By decreasing HBV replication, it is preferably meant that the HBV replication is decreased by at least 10 or 100 fold in comparison with the HBV replication in absence of treatment. For instance, the HBV replication can be assessed by determining the HBV DNA levels and this level is decreased by at least 10 or 100 fold in comparison with the HBV replication in absence of EYP001. Alternatively, HBV cccDNA level is decreased by at least 10, 15, 20, 25, 30, 35, 40, 45 or 50% in comparison with the absence of treatment.

As used herein, the terms “subject”, “individual” or “patient” are interchangeable and refer to a human, including adult, child, newborn and human at the prenatal stage. In a particular aspect, the subject or patient suffers of hepatitis B infection, in particular a chronic hepatitis B.

The terms “quantity,” “amount,” and “dose” are used interchangeably herein and may refer to an absolute quantification of a molecule.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “therapeutically effective amount” refers to a quantity of an active ingredient or of a pharmaceutical composition which prevents, removes or reduces the deleterious effects of the disease, particularly infectious disease. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “sub-therapeutic amount” or “sub-therapeutic dose” refers to a dosage which is less than that dosage which would produce a therapeutic result in the subject if administered in the absence of the other agent. For instance, “sub-therapeutic amount” or “sub-therapeutic dose” can refer to a dosage which is decreased by 25, 50, 70, 80 or 90% in comparison to the therapeutically effective amount, especially the conventional therapeutic dosage for the same indication and the same administration route when used alone. The conventional therapeutic dosages are those acknowledged by the drug approvals agencies (e.g., FDA or EMEA).

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

As used herein, the term “pegylated form” refers to a pegylated interferon.

By “a synergistic effect” is intended to refer to an effect for decreasing the HBV replication which is more than the sum of the effects of each molecule alone. HBV replication can be assessed by determining surface HBV antigen (HBsAg), HBeAg, HBV core related antigen (HBcrAg), HBV DNA, HBV pre-genomic RNA, HBV pre-core RNA and/or HBV cccDNA. More particularly, the effect is observed on the pre-genomic RNA (HBV pgRNA) and/or on the hepatitis B core related antigen (HBcrAg).

Combined Treatment

The present invention relates to the use of a combination of an FXR agonist and IFN for the treatment of hepatitis B virus infection, especially chronic hepatitis B. Indeed, this combination leads to a synergistic effect against HBV.

Accordingly, the present invention relates to
a pharmaceutical composition comprising an FXR agonist and IFN-α or a pegylated form thereof, and optionally a pharmaceutically acceptable carrier and/or an additional active ingredient, in particular for use in the treatment of hepatitis B virus infection, especially chronic hepatitis B, wherein the FXR agonist and IFN-α are used so as to obtain a synergistic effect for decreasing the HBV replication;
a product or kit containing an FXR agonist or a pharmaceutical composition comprising it and IFN-α or a pegylated form thereof as a combined preparation for simultaneous, separate or sequential use, in particular in the treatment of hepatitis B virus infection, especially chronic hepatitis B, wherein the FXR agonist and IFN-α are used so as to obtain a synergistic effect for decreasing the HBV replication; optionally, the product or kit may comprise at least one additional active ingredient;
a combined preparation which comprises an FXR agonist or a pharmaceutical composition comprising it and IFN-α or a pegylated form thereof for simultaneous, separate or sequential use, in particular in the treatment of hepatitis B virus infection, especially chronic hepatitis B, wherein the FXR agonist and IFN-α are used so as to obtain a synergistic effect for decreasing the HBV replication; optionally, the combined preparation may comprise at least one additional active ingredient;
a pharmaceutical composition comprising an FXR agonist for the use in the treatment of hepatitis B virus infection, especially chronic hepatitis B, in combination with a treatment with IFN-α or a pegylated form thereof, wherein the FXR agonist and IFN-α are used so as to obtain a synergistic effect for decreasing the HBV replication; optionally, the pharmaceutical composition may comprise at least one additional active ingredient;
a pharmaceutical composition comprising IFN-α or a pegylated form thereof for the use in the treatment of hepatitis B virus infection, especially chronic hepatitis B, in combination with a treatment with an FXR agonist, wherein the FXR agonist and IFN-α are used so as to obtain a synergistic effect for decreasing the HBV replication; optionally, the pharmaceutical composition may comprise at least one additional active ingredient;
the use of a pharmaceutical composition comprising an FXR agonist for the manufacture of a medicament for the treatment of hepatitis B virus infection, especially chronic hepatitis B, in combination with a treatment with IFN-α or a pegylated form thereof, wherein the FXR agonist and IFN-α are used so as to obtain a synergistic effect for decreasing the HBV replication; optionally, the pharmaceutical composition may comprise at least one additional active ingredient;
the use of a pharmaceutical composition comprising IFN-α or a pegylated form thereof for the manufacture of a medicament for the treatment of hepatitis B virus infection, especially chronic hepatitis B, in combination with a treatment with an FXR agonist, wherein the FXR agonist and IFN-α are used so as to obtain a synergistic effect for decreasing the HBV replication; optionally, the pharmaceutical composition may comprise at least one additional active ingredient;
the use of a pharmaceutical composition comprising an FXR agonist and IFN-α or a pegylated form thereof, and optionally a pharmaceutically acceptable carrier for the manufacture of a medicament for the treatment of hepatitis B virus infection, especially chronic hepatitis B, wherein the FXR agonist and IFN-α are used so as to obtain a synergistic effect for decreasing the HBV replication; optionally, the pharmaceutical composition may comprise at least one additional active ingredient;
a method for treating hepatitis B virus infection, especially chronic hepatitis B, in a subject in need thereof, comprising administering an effective amount of a pharmaceutical composition comprising a) an FXR agonist, b) IFN-α or a pegylated form thereof, and a pharmaceutically acceptable carrier, wherein the FXR agonist and IFN-α are used so as to obtain a synergistic effect for decreasing the HBV replication; optionally, the pharmaceutical composition may comprise at least one additional active ingredient or the method may further comprise the administration of at least one additional active ingredient;
a method for treating hepatitis B virus infection, especially chronic hepatitis B, in a subject in need thereof, comprising administering an effective amount of a pharmaceutical composition comprising an FXR agonist, and an effective amount of a pharmaceutical composition comprising IFN-α or a pegylated form thereof, wherein the FXR agonist and IFN-α are used so as to obtain a synergistic effect for decreasing the HBV replication; optionally, one of the pharmaceutical compositions may comprise at least one additional active ingredient or the method may further comprise the administration of at least one additional active ingredient.

FXR agonists are well known to the skilled person.

For example, the skilled person may easily identify FXR agonist from the following publications (the disclosure of which being incorporated herein by reference):
Abenavoli L, et al. Pharmaceuticals (Basel). 2018 Oct. 11; 11(4). pii: E104. doi: 10.3390/ph11040104. Review.
Adorini L, et al. Drug Discov Today. 2012 September; 17(17-18):988-97. doi: 10.1016/j.drudis.2012.05.012. Epub 2012 May 29. Review.
Akwabi-Ameyaw A, et al. Bioorg Med Chem Lett. 2009 Aug. 15; 19(16):4733-9. doi: 10.1016/j.bmcl.2009.06.062. Epub 2009 Jun. 21.
Akwabi-Ameyaw A, et al. Bioorg Med Chem Lett. 2008 Aug. 1; 18(15):4339-43. doi: 10.1016/j.bmcl.2008.06.073. Epub 2008 Jun. 28.
Akwabi-Ameyaw A, et al. Bioorg Med Chem Lett. 2011 Oct. 15; 21(20):6154-60. doi: 10.1016/j.bmcl.2011.08.034. Epub 2011 Aug. 11.
Baghdasaryan A, et al. Hepatology. 2011 October; 54(4):1303-12. doi: 10.1002/hep.24537.
Bass J Y, et al. Bioorg Med Chem Lett. 2009 Jun. 1; 19(11):2969-73. doi: 10.1016/j.bmcl.2009.04.047. Epub 2009 Apr. 18.
Bass J Y, et al. Bioorg Med Chem Lett. 2011 Feb. 15; 21(4):1206-13. doi: 10.1016/j.bmcl.2010.12.089. Epub 2010 Dec. 23.
Buijsman et al., Curr. Med. Chem. 2005, 12, 1017
Carino et al, Sci Rep. 2017 Feb. 16; 7:42801. doi: 10.1038/srep42801.
Chiang P C, et al. J Pharm Sci. 2011 November; 100(11):4722-33. doi: 10.1002/jps.22664. Epub 2011 Jun. 9.
Crawley, Expert Opin. Ther. Pat. 2010, 20, 1047
Feng S, et al. Bioorg Med Chem Lett. 2009 May 1; 19(9):2595-8. doi: 10.1016/j.bmcl.2009.03.008. Epub 2009 Mar. 9.
Festa et al, Front Pharmacol. 2017 Mar. 30; 8:162. doi: 10.3389/fphar.2017.00162. eCollection 2017.
Finamore et al, Sci Rep. 2016 Jul. 6; 6:29320. doi: 10.1038/srep29320.
Flatt B, et al. J Med Chem. 2009 Feb. 26; 52(4):904-7. doi: 10.1021/jm8014124.
Gege et al, Curr Top Med Chem. 2014; 14(19):2143-58.
Gege et al, Handbook of Experimental Pharmacology, doi: 10.1007/164_2019_232.
Genin et al, J Med Chem. 2015 Dec. 24; 58(24):9768-72. doi: 10.1021/acs.jmedchem.5b01161. Epub 2015 Dec. 2.
Ghebremariam Y T, et al. PLoS One. 2013 Apr. 4; 8(4):e60653. doi: 10.1371/journal.pone.0060653. Print 2013.
Gioiello A, et al. Bioorg Med Chem. 2011 Apr. 15; 19(8):2650-8. doi: 10.1016/j.bmc.2011.03.004. Epub 2011 Mar. 10.
Hoekstra M, et al. Mol Cell Endocrinol. 2012 Oct. 15; 362(1-2):69-75. doi: 10.1016/j.mce.2012.05.010. Epub 2012 May 27.
Iguchi Y, et al. Steroids. 2010 January; 75(1):95-100. doi: 10.1016/j.steroids.2009.11.002. Epub 2009 Nov. 12. Kinzel et al, Bioorg Med Chem Lett. 2016 Aug. 1; 26(15):3746-53. doi: 10.1016/j.bmcl.2016.05.070. Epub 2016 May 24.
Lin H R. Bioorg Med Chem Lett. 2012 Jul. 15; 22(14):4787-92. doi: 10.1016/j.bmcl.2012.05.057. Epub 2012 May 23.
Lundquist J T, et al. J Med Chem. 2010 Feb. 25; 53(4):1774-87. doi: 10.1021/jm901650u.
Ma Y, et al. Pharm Res. 2013 May; 30(5):1447-57. doi: 10.1007/511095-013-0986-7. Epub 2013 Feb. 1.
Marinozzi M, et al. Bioorg Med Chem. 2013 Jul. 1; 21(13):3780-9. doi: 10.1016/j.bmc.2013.04.038. Epub 2013 Apr. 23.
Massafra et al. Pharmacol Ther. 2018 November; 191:162-177. doi: 10.1016/j.pharmthera.2018.06.009. Epub 2018 Jun. 20.
Misawa T, et al. Bioorg Med Chem Lett. 2012 Jun. 15; 22(12):3962-6. doi: 10.1016/j.bmcl.2012.04.099. Epub 2012 Apr. 30.
Pellicciari et al, J Med Chem. 2016 Oct. 4.
Richter H G, et al. Bioorg Med Chem Lett. 2011 Feb. 15; 21(4):1134-40. doi: 10.1016/j.bmcl.2010.12.123.
Epub 2010 Dec. 31.
Rizzo G, et al. Mol Pharmacol. 2010 October; 78(4):617-30. doi: 10.1124/mo1.110.064501. Epub 2010 Jul. 14.
Roda et al, J Pharmacol Exp Ther. 2014 July; 350(1):56-68. doi: 10.1124/jpet.114.214650. Epub 2014 May 1.
Schuster D, et al. Bioorg Med Chem. 2011 Dec. 1; 19(23):7168-80. doi: 10.1016/j.bmc.2011.09.056. Epub 2011 Oct. 4.
Schwabl et al, J Hepatol. 2017 April; 66(4):724-733. doi: 10.1016/j.jhep.2016.12.005. Epub 2016 Dec. 18.
Samlley et al, Bioorg Med Chem Lett. 2015 Jan. 15; 25(2):280-4. doi: 10.1016/j.bmcl.2014.11.050. Epub 2014 Nov. 26.
Sepe et al. Expert Opin Ther Pat. 2018 May; 28(5):351-364. doi: 10.1080/13543776.2018.1459569. Epub 2018 Apr. 13. Review.
Sepe et al. Expert Opin Ther Pat. 2015; 25(8):885-96. doi: 10.1517/13543776.2015.1045413. Review.
Soisson S M, et al. Proc Natl Acad Sci USA. 2008 Apr. 8; 105(14):5337-42. doi: 10.1073/pnas.0710981105. Epub 2008 Apr. 7.
Townsend S A, Newsome P N. Aliment Pharmacol Ther. 2017 September; 46(5):494-507. doi: 10.1111/apt.14210. Epub 2017 Jul. 4.
Tully et al, J Med Chem. 2017 Dec. 28; 60(24):9960-9973. doi: 10.1021/acs.jmedchem.7b00907. Epub 2017 Dec. 8.
Wang et al, J Am Soc Nephrol. 2018 January; 29(1):118-137. doi: 10.1681/ASN.2017020222. Epub 2017 Oct. 31.
Wang et al, Bioorg Med Chem Lett. 2017 Aug. 1; 27(15):3386-3390. doi: 10.1016/j.bmcl.2017.06.003. Epub 2017 Jun. 3.
Wang H, et al. Expert Opin Ther Pat. 2018 November; 28(11):765-782. doi: 10.1080/13543776.2018.1527906. Epub 2018 Oct. 8. Review
Watanabe M, et al. J Biot Chem. 2011 Jul. 29; 286(30):26913-20. doi: 10.1074/jbc.M111.248203. Epub 2011 Jun. 1.
Yu D, et al. Steroids. 2012 November; 77(13):1335-8. doi: 10.1016/j.steroids.2012.09.002. Epub 2012 Sep. 21.
Zhang S, et al. J Hepatol. 2009 August; 51(2):380-8. doi: 10.1016/j.jhep.2009.03.025. Epub 2009 May 18.

Typically, FXR agonists include the class of steroid FXR agonists and non-steroid FXR agonists.

In certain embodiments of the invention, the FXR agonist is selected from small molecule compounds which act as FXR modulators that have been disclosed in the following publications: EP1392714; EP1568706; JP2005281155; US20030203939; US2005080064; US2006128764; US20070015796; US20080038435; US20100184809; US20110105475; U.S. Pat. No. 6,984,560; WO2000037077; WO200040965; WO200076523; WO2003015771; WO2003015777; WO2003016280; WO2003016288; WO2003030612; WO2003016288; WO2003080803; WO2003090745; WO2004007521; WO2004048349; WO2004046162; WO2004048349; WO2005082925; WO2005092328; WO2005097097; WO2007076260; WO2007092751; WO2007140174; WO2007140183; WO2008002573; WO2008025539; WO2008025540; WO200802573; WO2008051942; WO2008073825; WO2008157270; WO2009005998; WO2009012125; WO2009027264; WO2009080555; WO2009127321; WO2009149795; WO2010028981; WO2010034649; WO2010034657; WO2017218330; WO2017218379; WO2017201155; WO2017201152; WO2017201150; WO2017189652; WO2017189651; WO2017189663; WO2017147137; WO2017147159; WO2017147174; WO2017145031; WO2017145040; WO2017145041; WO2017133521; WO2017129125; WO2017128896; WO2017118294; WO2017049172; WO2017049176; WO2017049173; WO2017049177; WO2016173397; WO2016173493; WO2016168553; WO2016161003; WO2016149111; WO2016131414; WO2016130809; WO2016097933; WO2016096115; WO2016096116; WO2016086115; WO2016073767; WO2015138986; WO2018152171; WO2018170165, WO2018170166, WO2018170173, WO2018170182, WO2018170167; WO2017078928; WO2014184271; WO2013007387; WO2012087519; WO2011020615; WO2010069604; WO2013037482; US2017275256; WO2005080064; WO2018190643; WO2018215070; WO2018215610; WO2018214959; WO2018081285; WO2018067704; WO2019007418; WO2018059314; WO2017218337; WO2020231917; WO2020211872; WO2020168143; WO2020168148; WO2020156241; WO2020150136; WO2020114307; WO2020061118; WO2020061114; WO2020061112; WO2020061113; WO2020061116, WO2020061117; WO2020011146; WO2020001304; WO2019160813; WO2019120088; WO2019118571; WO2019089667; WO2019089672; WO2019089665; WO2019089664; WO2019089670; the disclosure of which being incorporated herein by reference.

In an aspect, the FXR agonist can be any FXR agonists disclosed in the following patent applications: WO2017/049172, WO2017/049176, WO2017/049173, WO2017/049177, WO2018/170165, WO2018/170166, WO2018/170173, WO2018/170182, and WO2018/170167.

Specific examples of FXR agonists include but are not limited to EYP001, GW4064 (as disclosed in PCT Publication No. WO 00/37077 or in US2007/0015796), 6-ethyl-chenodeoxycholic acids, especially 3α, 7α-dihydroxy 7α-dihydroxy-6α-ethyl-5β-cholan-24-oic acid, also referred to as INT-747 (OCA); INT-777; 6-ethyl-ursodeoxycholic acids, INT-1103, UPF-987, WAY-362450, MFA-1, GW9662, T0901317, fexaramine, 3β-azido-6α-ethyl-7α-hydroxy-5β-cholan-24-oic acid, GS-9674 (Cilofexor) (Phenex Pharmaceuticals AG), Tropifexor (LJN452), LMB763 (Nidufexor), PX-102 (PX-20606), PX-104 (Phenex 104), EDP-297, EDP-305, TERN-101 (LY2562175), MET-409, MET-642, WAY362450, Fexaramine, in particular fexaramine-3 (Fex-3), AGN-242266 (former AKN-083, Allergan), BAR502, BAR704, PX20606, PX20350, 3α,7α,11β-Trihydroxy-6α-ethyl-5β-cholan-24-oic Acid (TC-100), 6-(4-{[5-Cyclopropyl-3-(2,6-dichlorophenyl)isoxazol-4-yl]methoxy}piperidin-1-yl)-1-methyl-1H-indole-3-carboxylic Acid, 3,6-dimethyl-1-(2-methylphenyl)-4-(4-phenoxyphenyl)-4,8-dihydro-1H-pyrazolo[3,4-e][1,4]thiazepin-7-one; obeticholic acid, a cholic acid, a deoxycholic acid, a glycocholic acid, a glycodeoxycholic acid, a taurocholic acid, a taurodihydrofusidate, a taurodeoxycholic acid, a cholate, a glycocholate, a deoxycholate, a taurocholate, a taurodeoxycholate, a chenodeoxycholic acid, an ursodeoxycholic acid, a tauroursodeoxycholic acid, a glycoursodeoxycholic acid, a 7-B-methyl cholic acid, a methyl lithocholic acid, GSK-8062 (CAS No. 943549-47-1). In some embodiments, the FXR agonist is selected from natural bile acids, preferably chenodeoxycholic acid [CDCA] or taurine- or glycine-conjugated CDCA [tauro-CDCA or glyco-CDCA] and synthetic derivatives of natural bile acids, preferably 6-Ethyl-CDCA or taurine- or glycine-conjugated 6-Ethyl-CDCA, natural non-steroidal agonists, preferably Diterpenoids such as Cafestol and Kahweol, or synthetic non-steroidal FXR agonists.

In some embodiments, the FXR agonist is selected from the group consisting of obeticholic acid (Intercept Pharma), cholic acid (CT-RS); GS-9674 (Cilofexor) (Phenex Pharmaceuticals AG), Tropifexor (LJN452) (Novartis Pharmaceuticals), LMB763 (Nidufexor), PX-102 (PX-20606), PX-104 (Phenex 104), EYP001, OCA, EDP-297, EDP-305, a steroidal non-carboxylic acid FXR agonist (Enanta Pharmaceuticals), Turofexorate Isopropyl (Pfizer), INT-767 (Intercept Pharmaceuticals), LY-2562175 (Lilly), AGN-242266 (former AKN-083, Allergan), EP-024297 (Enanta Pharmaceuticals), M-480 (Metacrine), TERN-101 (LY2562175), MET-409 (Metacrine), MET-642 (Metacrine), BAR502, RDX-023 (Ardelyx), GW4064, GW6046, WAY362450, Cafestol, Fexaramine and the compound PXL007 (also named EYP001 or EYPOO1a) identified by the CAS No. 1192171-69-9 (described in WO 2009127321). In a particular embodiment, the FXR agonist is selected from the group consisting of INT-747, the compound identified by EDP-305 a steroidal non-carboxylic acid FXR agonist (Enanta Pharmaceuticals) and the compound identified by the CAS No. 1192171-69-9 (described in WO 2009127321).

In a particular aspect, the FXR agonist is selected from the group consisting of LIN452 (Tropifexor), GS-9674 (Cilofexor), LMB763 (Nidufexor), PX-102 (PX-20606), PX-104 (Phenex 104), OCA (Ocaliva), EDP-297, EDP-305, TERN-001, MET-409, MET-642, GW4064, WAY362450 (Turofexorate isopropyl), Fexaramine, AGN242266 (AKN-083), BAR502 and PXL007 (also named EYP001).

In a very particular aspect, the FXR agonist is selected from the group consisting of OCA (Ocaliva) (Intercept), EDP-297 (Enanta), EDP-305 (Enanta), GS-9674 (Cilofexor) (Gilead), TERN-001 (TERNS), MET-409 (Metacrine), MET-642 (Metacrine), UN452 (Tropifexor) (Novartis), LMB763 (Nidufexor) (Novartis), and AGN242266 (AKN-083) (Abbvie).

In a particular aspect, the FXR agonist is selected from the group consisting of the compound disclosed in Table 1.

TABLE 1

| | |
|---|---|
| LJN452 (Tropifexor) Cas Number 1383816-29-2 2-(3-((5-cyclopropyl-3-(2- (trifluoromethoxy)phenyl)isoxazol-4- yl)methoxy)-8-azabicyclo[3.2.1]octan-8-yl)-4- fluorobenzo[d]thiazole-6-carboxylic acid | 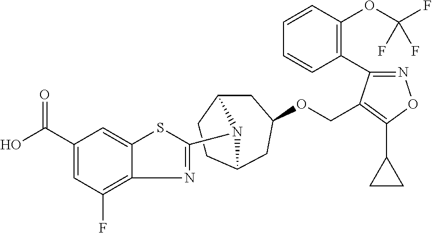 [SMILES: O=C(O)c1cc(F)c2nc(N3[C@@H]4CC[C@H]3C[C@@H](OCc3c(-c5ccccc5OC(F)(F)F)noc3C3CC3)C4)sc2c1] |
| LMB763 (Nidufexor) Cas Number 1773489-72-7 4-[(N-benzyl-8-chloro-1-methyl-1,4- dihydro[1]benzopyrano[4,3-c]pyrazole-3- carboxamido)methyl]benzoic acid | 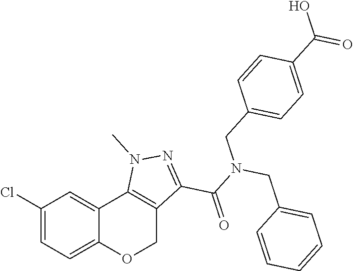 [SMILES: Cn1nc(C(=O)N(Cc2ccccc2)Cc2ccc(C(=O)O)cc2)c2c1-c1cc(Cl)ccc1OC2] |
| GS-9674 (Cilofexor) Cas Number 1418274-28-8 2-[3-[2-Chloro-4-[[5-cyclopropyl-3-(2,6- dichlorophenyl)-4- isoxazolyl]methoxy]phenyl]-3-hydroxy-1- azetidinyl]-4-pyridinecarboxylic acid | 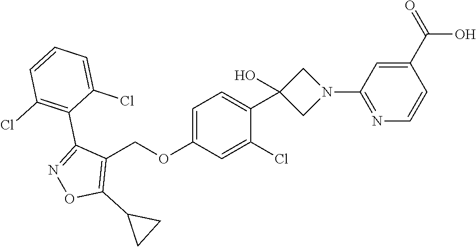 [SMILES: O=C(O)c1ccnc(N2CC(O)(c3ccc(OCc4c(-c5c(Cl)cccc5Cl)noc4C4CC4)cc3Cl)C2)c1] |
| PX-102 (PX-20606) Cas Number 1268244-85-4 4-(2-(2-Chloro-4-((5-cyclopropyl-3-(2,6- dichlorophenyl)isoxazol-4- yl)methoxy)phenyl)cyclopropyl)benzoic acid | 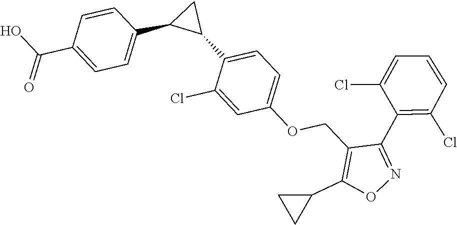 [SMILES: O=C(O)c1ccc([C@H]2C[C@@H]2c2ccc(OCc3c(-c4c(Cl)cccc4Cl)noc3C3CC3)cc2Cl)cc1] |
| PX-104 or Phenex 104 | enantiomer of PX-102 |
| OCA (Ocaliva or INT-747) Cas Number 459789-99-2 Cholan-24-oic acid, 6-ethyl-3,7-dihydroxy-, (3α,5β,6α,7α)- | 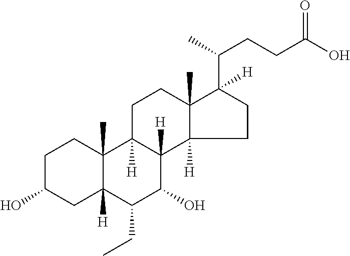 |
| EDP-305 Cas Number 1933507-63-1 Benzenesulfonamide, 4-(1,1-dimethylethyl)- N-[[[(3α,5β,6α,7α)-6-ethyl-3,7-dihydroxy-24- norcholan-23-yl]amino]carbonyl]- | 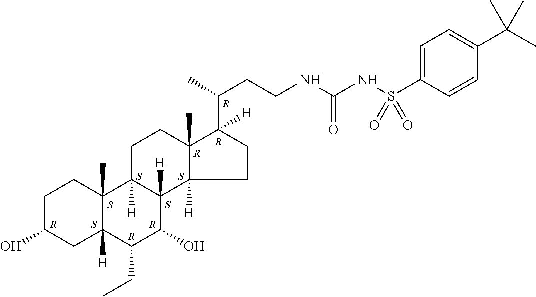 |
| TERN-101 (LY2562175) Cas Number 1103500-20-4 6-(4-{[5-Cyclopropyl-3-(2,6- dichlorophenyl)isoxazol-4- yl]methoxy}piperidin-1-yl)-1-methyl-1H- indole-3-carboxylic acid | 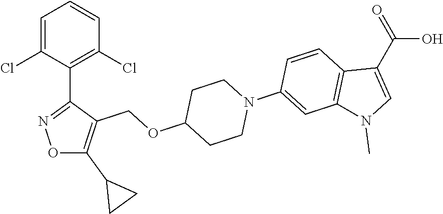 [SMILES: Cn1cc(C(=O)O)c2ccc(N3CCC(OCc4c(-c5c(Cl)cccc5Cl)noc4C4CC4)CC3)cc21] |
| MET409 | Developed by Metacrine |
| MET642 | Disclosed in WO2017049173 |
| | 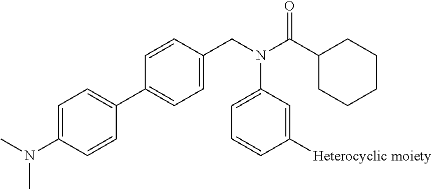 |
| GW4064 Cas Number 278779-30-9 3-[2-[2-Chloro-4-[[3-(2,6-dichlorophenyl)-5- (1-methylethyl)-4- isoxazolyl]methoxy]phenyl]ethenyl]benzoic acid | 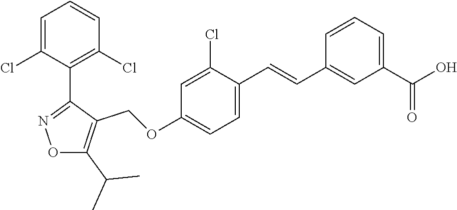 [SMILES: CC(C)c1onc(-c2c(Cl)cccc2Cl)c1COc1ccc(/C=C/c2cccc(C(=O)O)c2)c(Cl)c1] |
| WAY362450 (Turofexorate isopropyl or XL335 or FXR450) Cas Number 629664-81-9 3-(3,4-Difluoro-benzoyl)-1,1-dimethylene- 1,2,3,6-tetrahydro-azepino [4,5-b]indole-5- carboxylic acid isopropyl ester, 3-(3,4- Difluorobenzoyl)-1,2,3,6-tetrahydro-1,1- dimethyl-azepino[4,5-b]indole-5-carboxylic acid 1-methylethyl ester, | 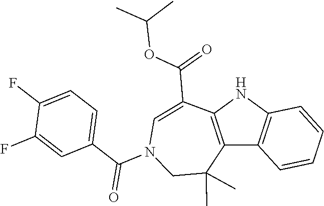 [SMILES: CC(C)OC(=O)C1=CN(C(=O)c2ccc(F)c(F)c2)CC(C)(C)c2c1[nH]c1ccccc21] |
| Fexaramine Cas Number 574013-66-4 3-[3-[(Cyclohexylcarbonyl)[[4'- (dimethylamino) [1,1'-biphenyl]-4- yl]methyl]amino]phenyl]-2-propenoic acid methyl ester | 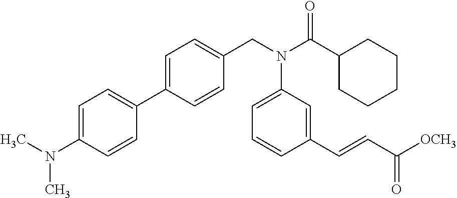 [SMILES: COC(=O)/C=C/c1cccc(N(Cc2ccc(-c3ccc(N(C)C)cc3)cc2)C(=O)C2CCCCC2)c1] |
| AGN242266 (AKN-083) | 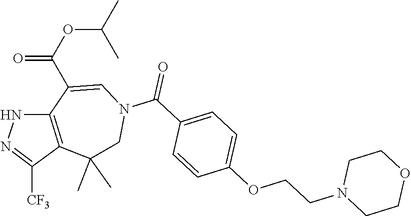 [SMILES: CC(C)OC(=O)C1=CN(C(=O)c2ccc(OCCN3CCOCC3)cc2)CC(C)(C)c2c(C(F)(F)F)n[nH]c21] |
| BAR502 Cas Number 1612191-86-2 6α-ethyl-3α, 7α-dihydroxy-24-nor-5ß-cholan- 23-ol | 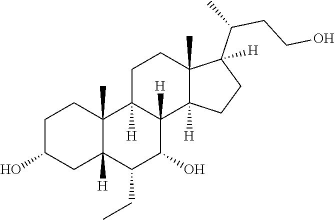 |
| EYP001 Cas Number 1192171-69-9 | 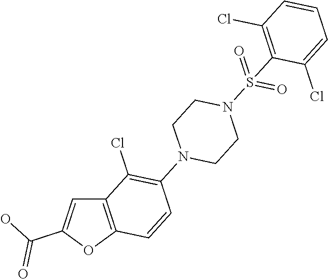 [SMILES: O=C(O)c1cc2c(Cl)c(N3CCN(S(=O)(=O)c4c(Cl)cccc4Cl)CC3)ccc2o1] |

and any pharmaceutically acceptable salt thereof.

In a particular aspect, the FXR agonist is selected from the group consisting of Tropifexor, Nidufexor, Ocaliva and GW4064 or any pharmaceutically acceptable salt thereof.

In a preferred aspect of the invention, the FXR agonist is not EYP001.

The FXR agonist can be administered once, twice or three times a day, preferably once or twice, for example in the morning (e.g., between 6 and 10 am) or in the evening (e.g., 6 and 10 pm). In one aspect, the FXR agonist is administered once a day. In another aspect, the FXR agonist is administered twice a day. It is preferably administered every day. However, an administration every 2, 3, 4, 5, 6 or 7 days can also be contemplated. The daily dosage of the FXR agonist may be varied over a wide range from 1 μg to 1,000 mg per adult per day. The FXR agonist can be administered by oral, sublingual, subcutaneous, intramuscular, intravenous, transdermal, local or rectal administration, preferably for oral administration.

In a particular aspect, the FXR agonist is to be administered at a sub-therapeutic amount. Optionally, the dosage of the FXR agonist could be reduced at least by a factor of 2, 3, 4 or 5, preferably at least by a factor 2 or 3. Optionally, the dosage of the FXR agonist could be in the range from 0.001 to 200 mg per day or from 50 to 200 mg per day or from 50 to 100 mg per day.

In a particular aspect, the dosage of the FXR agonist is a dosage which is decreased by 25, 50, 70, 80 or 90% in comparison to the therapeutically effective amount, especially the conventional therapeutic dosage for the same indication and the same administration route when used alone.

The IFN-α can be for instance IFN-α1 or IFN-α2, e.g., IFN-α1a, IFN-α1b, IFN-α2a, IFN-α2b, IFN-α2c or consensus IFN-α. In a very particular aspect, IFN is IFN-α2a, IFN-α2b or a pegylated form thereof.

Optionally, IFN-α is selected from the non-exhaustive list consisting of consensus IFN-α (e.g., INFERGEN®, Locteron®, IFN-α1b (e.g., HAPGEN®), IFN-α2a (Roferon-A®, MOR-22, Inter 2A, Inmutag, Inferon), a pegylated IFN-α2a (e.g., PEGASYS®, YPEG-IFNα-2a, PEG-INTRON®, Pegaferon), IFN-α2b (e.g., INTRON A®, Alfarona, Bioferon, Inter 2B, citpheron, Zavinex, Ganapar, etc. . . . ), a pegylated IFN-α2b (e.g., Pegintron®, Albuferon, AOP2014/P1101, Algeron, Pai Ge Bin), and IFN-α2c (e.g. Berofor Alpha). In a particular aspect, IFN is a pegylated IFN-α2a (e.g., PEGASYS®) or a pegylated IFN-α2b (Pegintron®).

In an aspect, the IFNα or a pegylated form thereof is administered by subcutaneous route once a week; for instance, at a dosage varying from 1 μg to 500 μg, preferably from 10 μg to 500 μg, still more preferably from 100 μg to 250 μg, such as 100, 110, 120, 130, 140, 150, 160, 170, 180, 190 or 200 μg.

Optionally, the IFNα or a pegylated form thereof can be administered at a sub-therapeutic amount.

Optionally, the IFNα or a pegylated form thereof and the FXR agonist are administered at a sub-therapeutic amount.

Preferably, the combined therapy comprising an FXR agonist and IFN-α is effective for decreasing the replication of HBV.

In the context of a combined treatment with an FXR agonist and IFN-α (i.e., IFN-α2a, IFN-α2b or a pegylated form thereof), the inventors surprisingly observed a synergistic effect is at least twice stronger when EYP001 is administered once a day in comparison with an administration twice a day with the same daily dosing. In addition, the inventors observed that, surprisingly, fewer pruritus occurs when EYP001 is administered once a day rather than twice a day. Therefore, in a particular aspect, the FXR agonist is administered once a day.

Preferably, the composition, dosage unit or dosage form contains from 1 μg to 500 or 1000 mg of the FXR agonist for the symptomatic adjustment of the dosage to the patient to be treated.

In one aspect, the dosage form can be a scored dosage form. Alternatively, the daily dosage can be provided by administering several dosage forms.

The FXR agonist may be combined with pharmaceutically acceptable excipients, and optionally sustained-release matrices, such as biodegradable polymers, to form pharmaceutical compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The pharmaceutical compositions comprising an FXR agonist can be suitable for oral, sublingual, subcutaneous, intramuscular, intravenous, transdermal, local or rectal administration, preferably for oral administration.

The FXR agonist, alone or in combination with another active principle, can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

In a preferred embodiment, the oral dosage form is a capsule or a tablet. Optionally, the oral dosage form is a scored dosage form. Optionally, the dosage form can be scored into four pieces, three pieces or two pieces.

Optionally, the treatment lasts from 2-4 months up to 24 months, for instance between 2 and 24 months or between 2 and 12 months, e.g., 2, 3, 4, 5, 6, 7, 8, 9, 10, 11, 12, 13, 14, 15, 16, 17, 18, 19, 20, 21, 22, 23 or 24 months. In a very specific aspect, the treatment lasts from 12 to 52 weeks, preferably from 45 to 52 weeks, for instance 48 weeks.

The FXR agonist and IFN-α or a pegylated form thereof can be used in combination with at least one additional active ingredient. Preferably, the additional active ingredient is an antiviral, more particularly an antiviral having an activity against HBV. In a preferred aspect, the at least one additional active ingredient is a polymerase inhibitor selected from the group consisting of L-nucleosides, deoxyguanosine analogs and nucleoside phosphonates. In a very specific aspect, the at least one additional active ingredient is selected from the group consisting of lamivudine, telbivudine, emtricitabine, entecavir, adefovir and tenofovir.

Further aspects and advantages of the present invention will be described in the following examples, which should be regarded as illustrative and not limiting.

### EXAMPLES

### Example 1—FXR Agonist EYP001

HBV chronically infected patients (male (n=39) and female (n=34)) underwent a 4-week treatment course as either daily oral FXR agonist EYP001a monotherapy or placebo or entecavir (ETV) in Part A of the study (n=48) or in combination with interferon (n=23, weekly sub-cutaneous injections of pegylated IFNα2a, PEG-IFN) in Part B. Patient characteristics were: mean age of 39.7 years (range: 19-63); 6 of 73 were HBeAg-positive; 70% treatment naïve; mean baselines HBV DNA 4.2 (±1.5 SD) log 10 IU/mL, HBsAg 3.5 (±0.8 SD) log 10 IU/mL, and genotypes A (25), B(8), C(10), D(7) and E(4). Detailed virology characteristics are summarized in Table B and C. FXR engagement with all EYP001 doses led to decreases in C4 and increases in FGF19 (data not shown).

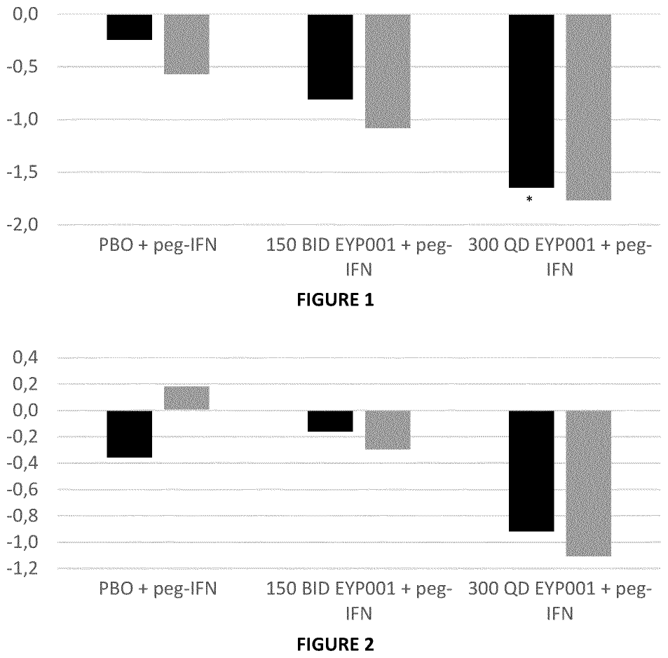

At end of treatment on day 29, 400 mg QD EYP001 decrease the mean HBsAg by −0.1 log 10 IU/mL (p<0.05). Surprisingly early markers of HBV replication pgRNA and HBcrAg showed a synergistic decrease when EYP001 was combined with peg-IFN, but not with peg-IFN or EYP001 monotherapies (Table A). The mean HBV pgRNA decrease was −1.7 log 10 Copies/mL (p<0.05) and the mean HBcrAg decrease was −0.9 log 10 IU/mL (p=0.15), whereas the Placebo+Peg-IFN group had no significant decline (−0.2 log 10 Copies/mL pgRNA, −0.4 log 10 IU/mL HBcrAg). This effect lasted at day 35, i.e. 7 days after end of treatment (EoT, FIG. 1 and FIG. 2). A stronger synergistic effect is observed with QD in comparison to BID.

TABLE A

| | HBVpgRNA Log10 Copies mL | | | | HBcrAg Log10 IU mL | | |
|---|---|---|---|---|---|---|---|
| Treatment | n | Mean | SD | P-value (a) | Mean | SD | P-value (a) |
| ETV (open-label 0.5 mg/day) | 7 | −0.3 | 2.0 | 0.67 | 0.03 | 0.6 | 0.91 |
| EYP001a (1 × 100 mg/day) | 7 | 0.5 | 1.2 | 0.33 | −0.3 | 1.1 | 0.45 |
| EYP001a (1 × 200 mg/day) | 8 | 0.4 | 1.7 | 0.59 | 0.2 | 1.1 | 0.56 |
| EYP001a (1 × 400 mg/day) | 8 | 0.2 | 1.4 | 0.65 | 0.02 | 0.1 | 0.50 |
| EYP001a (2 × 200 mg/day) | 7 | −0.1 | 1.9 | 0.92 | 1.2 | 2.2 | 0.19 |
| EYP001a (1 × 300 mg/day) + Peg-IFN | 8 | −1.7 | 1.9 | 0.04 * | −0.9 | 1.6 | 0.15 |
| EYP001a (2 × 150 mg/day) + Peg-IFN | 8 | −0.9 | 1.8 | 0.19 | −0.2 | 0.2 | 0.06 |
| Placebo + Peg-IFN | 8 | −0.2 | 1.0 | 0.52 | −0.4 | 0.9 | 0.30 |

* From Student paired test.

TABLE B
Summary of HBV infection parameters at baseline for Part A

| | EYP001a | | EYP001a | | EYP001a | | EYP001a | | EYP001a | | ETV 0.5 | | All | |
|---|---|---|---|---|---|---|---|---|---|---|---|---|---|---|
| | 1 × 100 mg | | 1 × 200 mg | | 1 × 400 mg | | 2 × 200 mg | | Total | | (mg/day) | | Subjects | |
| | (N = 7) | | (N = 8) | | (N = 9) | | (N = 9) | | (N = 33) | | (N = 7) | | (N = 48) | |
| ALT (measn, SD) | 36.2 | (20.8) | 28.6 | (11.0) | 22.3 | (6.1) | 32.4 | (12.5) | 29.7 | (13.5) | 24.0 | (10.9) | 28.4 | (12.4) |
| Treatment naive | 5 | (71%) | 6 | (75%) | 8 | (89%) | 8 | (89%) | 27 | (82%) | 4 | (57%) | 35 | (73%) |
| HBV DNA baseline mean | 4.76 | (1.15) | 3.46 | (0.44) | 3.84 | (0.77) | 4.35 | (1.9) | 4.08 | (1.26) | 3.82 | (0.70) | 4.03 | (1.20) |
| log10 IU/mL (SD) | | | | | | | | | | | | | | |
| HBsAg lgo10 IU/mL (SD) | 3.4 | (0.54) | 3.5 | (0.6) | 3.7 | (0.6) | 3.3 | (1.0 | 3.5 | (0.7) | 3.2 | (0.6) | 3.5 | (0.7) |
| HBV Genotype A | 2 | (29%) | 2 | (25%) | 2 | (22%) | 3 | (33%) | 9 | (27%) | 3 | (43%) | 12 | (25%) |
| HBV Genotype B or C or D or E | 3 | (xx %) | 5 | (xx %) | 5 | (%) | 3 | (%) | 16 | (%) | 1 | (14%) | 20 | (%) |
| HBeAg neg | 6 | (86%) | 7 | (88%) | 9 | (100%) | 7 | (78%) | 29 | (88%) | 7 | (100%) | 42 | (88%) |

TABLE C
Summary of HBV infection parameters at baseline for Part B

| | EYP001a | | EYP001a | | EYP001a + | | | | | |
|---|---|---|---|---|---|---|---|---|---|---|
| | (1 × 300 mg) + | | (2 × 150 mg) + | | PEG-IFN | | Placebo + | | All | |
| | PEG-IFN(18 μg) | | PEG-IFN(18 μg) | | Total | | PEG-IFN(18 μg) | | Subjects | |
| | (N = 8) | | (N = 9) | | (N = 17) | | (N = 8) | | (N = 25) | |
| ALT (mean, SD) | 34.7 | (38.6) | 27.3 | (13.5) | 30.4 | (26.2) | 32.2 | (12.4) | 31.0 | (22.6) |
| HBV DNA baseline mean | 4.17 | (1.79) | 4.26 | (1.80) | 4.22 | (1.74) | 4.87 | (2.49) | 4.42 | (1.98) |
| log10 IU/mL (SD) | | | | | | | | | | |
| HBsAg lgo10 IU/mL (SD) | 3.8 | (0.6) | 3.4 | (1.1) | 3.6 | (0.9) | 3.9 | (0.8) | 3.7 | (0.9) |
| HBV Treatment naive | 5 | (63%) | 8 | (89%) | 13 | (76%) | 3 | (38%) | 16 | (64%) |
| HBV Genotype A | 3 | (38%) | 1 | (11%) | 4 | (24%) | 4 | (50%) | 8 | (32%) |
| HBV Genotype B | 1 | (13%) | — | | 1 | (6%) | 2 | (25%) | 3 | (12%) |
| HBV Genotype C | 1 | (13%) | 2 | (22%) | 3 | (18%) | — | | 3 | (12%) |
| HBV Genotype D | — | | 1 | (11%) | 1 | (6%) | 1 | (13%) | 2 | (8%) |
| HBV Genotype E | 1 | (13%) | — | | 1 | (6%) | — | | 1 | (4%) |
| HBeAg neg | 7 | (88%) | 7 | (78%) | 14 | (82%) | 5 | (63%) | 19 | (76%) |
| HBeAg pos | | | 1 | (11%) | 1 | (6%) | 1 | (13%) | 2 | (8%) |
| anti-HBeAg pos | 7 | (88%) | 7 | (78%) | 14 | (82%) | 5 | (63%) | 19 | (76%) |

### Example 2—FXR Agonist GW4064

Material and Methods

Primary Human Hepatocytes

Primary human hepatocytes (PHH) were freshly prepared from human liver resection obtained from the Centre Léon Bérard (Lyon) with French ministerial authorizations (AC 2013-1871, DC 2013-1870, AFNOR NF 96 900 sept 2011) as previously described in Lecluyse et al (Methods Mol. Biol. Clifton NJ 640, 57-82 (2010)). Viruses

HBV stocks (genotype D, Genbank ID U95551) were prepared using the HepAD38 cell line according to previously described protocols in Ladner et al (Antimicrob. Agents Chemother. 41, 1715-1720 (1997)). Supernatants containing HBV particles were clarified (0.45 μm filter) and concentrated with 8% PEG 8000 (Sigma-Aldrich).

HBV DNA was quantified using the AmpliPrep/COBAS® TaqMan® HBV Test (Roche).

Chemicals

GW4064 [3-(2,6-dichlorophenyl)-4-(3-carboxy-2-chloro-stilben-4-yl)-oxymethyl-5-iso-propyl isoxazole] is a FXR agonist (EC50 90 nM), active both in vivo and in vitro (Maloney et al., J. Med. Chem. 43, 2971-2974 (2000). Although displaying a limited bioavailability, GW4064 has gained a widespread use as a powerful and selective FXR agonist and has reached the status of “reference compound” in this field. Interferon alpha-2 (ROFERON-A) was purchased from Roche.

HBs and HBe Quantification

HBs and HBe antigens secreted in cells supernatant were quantified, after required dilutions, with Autobio kits (AutoBio, China) according to manufacturer's protocol.

Quantification of Viral RNAs by qPCR

Total RNA was prepared using NucleoSpin RNA Plus (Macherey-Nagel). After DNA digestion with TURBO DNase (Ambion), maximum 1000 ng RNA were reverse-transcribed using High-Capacity RNA-to-cDNA kit (Thermo Fisher Scientific). Quantitative PCR was carried out with primers HBV-F (5′-AGCTACTGTGGAGTTACTCTCGT-3′ SEQ ID NO: 1) and HBV-R (5′-CAAAGAATTGCTTGCCTGAGTG-3′ SEQ ID NO: 2) for quantification of pregenomic/precore HBV RNA. cDNA was analysed by quantitative PCR (qPCR) using QuantiFast SYBR® Green PCR kit (Qiagen) on LightCycler® 480 instrument (Roche) using a 45 PCR cycles. All assays were performed in triplicate. Relative quantification was determined by normalizing the expression of each gene to S9 housekeeping gene using primers S9-F (5′-CCGCGTGAAGAGGAAGAATG-3′ SEQ ID NO: 3) and S9-R (5′-TTGGCAGGAAAACGAGACAAT-3′ SEQ ID NO: 4).

Results: Combined Treatment with FXR Modulator and IFN-α Synergistically Inhibits HBV Replication in PHH.

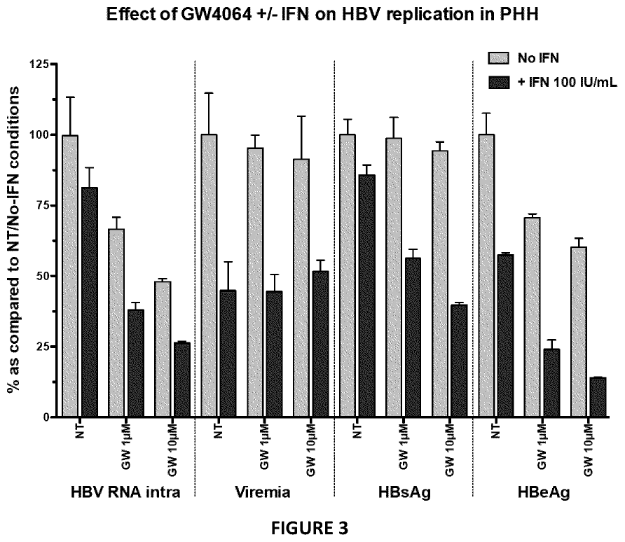

To determine the combined impact of FXR agonist and interferon-alpha (IFN-α) on HBV infection, in vitro infections were performed in primary human hepatocytes (PHH). PHH are naturally susceptible to infection with HBV virions produced in vitro, leading to very high levels of replication of the virus. 2 concentrations of GW4064 and a single concentration of IFN-α were used to determine the combined impact of FXR agonist and interferon-alpha (IFN-α) on HBV replication (FIG. 3). When used alone GW4064 was only capable to reduce in a dose response manner intracellular HBV RNA level and HBeAg secretion; it was unable to significantly impact the viremia and HBsAg secretion. IFN-α alone at 100 IU/mL was able to reduce viremia and HBeAg secretion, whereas it had almost no effect on intracellular HBV RNA level and HBsAg secretion. The combination of both molecules resulted in a synergistic effect on intracellular HBV RNA level, as well as HBeAg and HBsAg secretion; the synergistic effect was particularly strong on HBsAg secretion.

Conclusions

A synergy of action between an FXR agonist (GW4064) and IFN-α was observed in the PHH model. This synergy of action was particularly obvious on HBsAg secretion.

### Example 3—Synergistic Effect with Additional FXR Agonists

Material and Methods

HepaRG

The HepaRG cell line derived from a human cellular hepato carcinoma can differentiate and regain many phenotypic traits of hepatocytes after 4 weeks of culture under defined conditions (Hantz, O. et al. J. Gen. Virol. 90, 127-135 (2009)). HepaRG cells were cultured, differentiated, and infected by HBV as previously described (Gripon, P. et al. Proc. Natl. Acad. Sci. U.S.A 99, 15655-15660 (2002); Alfaiate, D. et al. Antiviral Res. 136, 19-31 (2016)). Briefly, for differentiation, cells were maintained for 2 weeks in standard medium then for at least 2 weeks in standard medium supplemented with 1.8% DMSO. The composition of standard medium was the following: William's E medium supplemented with 10% HyCLone FetalClone II serum (Thermo Fisher Scientific), penicillin/streptomycin, L-glutamine, Insulin-Transferrin-Selenium (Gibco) and 50 μM hydrocortisone hemisuccinate.

Primary Human Hepatocytes

Primary human hepatocytes (PHH) were freshly prepared from human liver resection obtained from the Centre Léon Bérard (Lyon) with French ministerial authorizations (AC 2013-1871, DC 2013-1870, AFNOR NF 96 900 sept 2011) as previously described (Lecluyse, E. L. & Alexandre, E. Methods Mol. Biol. Clifton NJ 640, 57-82 (2010)).

Viruses

HBV stocks (genotype D, Genbank ID U95551) were prepared using the HepAD38 cell line according to previously described protocols (Ladner, S. K. et al. Antimicrob. Agents Chemother. 41, 1715-1720 (1997)). Supernatants containing HBV particles were clarified (0.45 μm filter) and concentrated with 8% PEG 8000 (Sigma-Aldrich).

HBV DNA was quantified using the AmpliPrep/COBAS® TaqMan® HBV Test (Roche).

HBs Quantification

HBs antigen secreted in cells supernatant were quantified, after required dilutions, with Autobio kits (AutoBio, China) according to manufacturer's protocol.

Results: Combined Treatments with FXR Agonists and IFN-α Synergistically Inhibit HBsAg Secretion in HBV Infected PHH and in HBV Infected HepaRG Cells.

To determine the combined impact of FXR agonists and interferon-alpha (IFN-α) on HBV infection, in vitro infections were performed in primary human hepatocytes (PHH) and in differentiated HepaRG cells (dHepaRG). PHH are naturally susceptible to infection with HBV virions produced in vitro, leading to very high levels of replication of the virus. After differentiation, HepaRG cells are also susceptible to infection with HBV virions produced in vitro, although the replication level is lower than that observed in PHH.

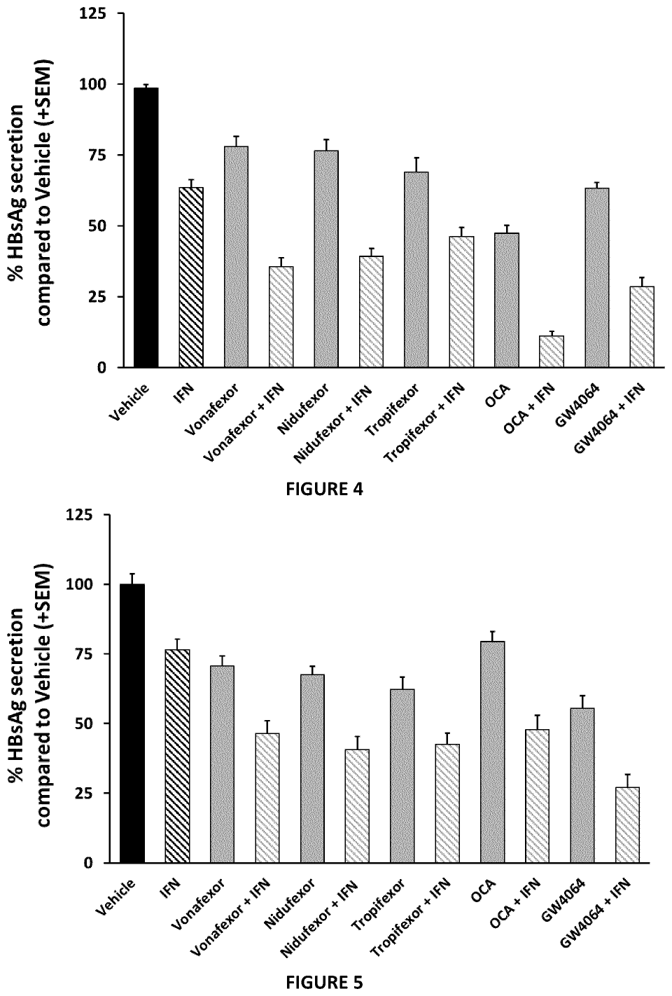

In the PHH model, five different FXR agonists (Vonafexor, Nidufexor, Tropifexor, OCA, and GW4064) were evaluated alone or in combination with IFN-α on HBsAg secretion (FIG. 4). The combination of each of the five FXR agonists with IFN-α resulted in a synergistic effect on HBsAg secretion, despite significant differences of structure between the tested FXR agonists.

The same type of synergistic effects between FXR agonism and IFN-α were also observed in the HepaRG model, with all the FXR agonists evaluated (FIG. 5).

Conclusions

A synergy of action on the inhibition of HBsAg secretion between different FXR agonists and IFN-α was observed in both the PHH and the HepaRG model. As the reduction of HBsAg level in the blood of chronically infected patients is considered as the next step for the improvement of HBV treatment in human, the results obtained are promising for the development of improved treatment of patients with a HBV chronic infection.

1-15. (canceled)

16. A method of treating a hepatitis B virus (HBV) infection in a subject comprising administering a farnesoid X receptor (FXR) agonist in combination with an interferon alpha (IFN-α), wherein the FXR agonist and IFN-α are administered so as to obtain a synergistic effect for decreasing HBV replication and wherein the FXR agonist is not EYP001.

17. The method according to claim 16, wherein the FXR agonist is selected from the group consisting of LJN452 (Tropifexor), LMB763 (Nidufexor), GS-9674 (Cilofexor), PX-102 (PX-20606), PX-104 (Phenex 104), OCA (Ocaliva), EDP-297, EDP-305, TERN-101 (LY2562175), MET-409, MET-642, GW4064, WAY362450 (Turofexorate isopropyl), Fexaramine, AGN242266 (AKN-083), and BAR502 or any pharmaceutically acceptable salt thereof.

18. The method according to claim 16, wherein the FXR agonist is selected from the group consisting of Tropifexor, Nidufexor, Ocaliva and GW4064, or any pharmaceutically acceptable salt thereof.

19. The method according to claim 16, wherein the FXR agonist is administered once a day.

20. The method according to claim 16, wherein the FXR agonist is administered twice a day.

21. The method according to claim 16, wherein the IFN-α is IFN-α2a, IFN-α2b or a pegylated form thereof.

22. The method according to claim 16, wherein the IFN-α is a pegylated IFN-α2a or a pegylated IFN-α2b.

23. The method according to claim 16, wherein the IFN-α is administered at a sub-therapeutic amount.

24. The method according to claim 16, wherein the IFN-α is administered by a subcutaneous route once a week.

25. The method according to claim 16, wherein the FXR agonist is administered at a sub-therapeutic amount.

26. The method according to claim 16, wherein the HBV infection is chronic hepatitis B infection.

27. The method according to claim 16, wherein the FXR agonist and the IFN-α are administered during a period of time from 5, 6, 7 or 8 weeks to 52 weeks.

28. The method according to claim 16, wherein the FXR agonist and the IFN-α are administered in combination with at least one additional active ingredient.

29. The method according to claim 28, wherein the at least one additional active ingredient is a polymerase inhibitor selected from the group consisting of L-nucleosides, deoxyguanosine analogs and nucleoside phosphonates.

30. The method according to claim 28, wherein the at least one additional active ingredient is selected from the group consisting of lamivudine, telbivudine, emtricitabine, entecavir, adefovir and tenofovir.


SEQUENCE LISTING (4 sequences)

SEQ ID NO: 1 - DNA, 23 bp, artificial
  primer
agctactgtggagttactctcgt

SEQ ID NO: 2 - DNA, 22 bp, artificial
  primer
caaagaattgcttgcctgagtg

SEQ ID NO: 3 - DNA, 20 bp, artificial
  primer
ccgcgtgaagaggaagaatg

SEQ ID NO: 4 - DNA, 21 bp, artificial
  primer
ttggcaggaaaacgagacaat